(19) United States
(12) Patent Application Publication
(10) Pub. No.: US 2024/0371504 A1
(43) Pub. Date: Nov. 7, 2024

(54) SELF-SERVICE STATION AND MEDICAL INFORMATION MANAGEMENT SYSTEM FOR SCREENING MEDICAL CONSULTATIONS

(71) Applicant: FOXCONN BRASIL INDUSTRIA E COMERCIO LTDA., Jundiaí (BR)

(72) Inventors: Henry JONG HWANG CHENG, Jundiaí (BR); Luis Eduardo PEDIGONI BULISANI, Jundiaí (BR)

(21) Appl. No.: 18/686,979

(22) PCT Filed: Nov. 9, 2021
(86) PCT No.: PCT/BR2021/050490

§ 371 Date: Feb. 27, 2024

(30) Foreign Application Priority Data
Sep. 1, 2021 (BR) BR 1020210173696

(51) Int. Cl. G16H 40/20; G16H 10/20; G16H 10/60; G16H 40/63; G16H 50/30

(52) U.S. Cl. CPC G16H 40/20; G16H 10/20; G16H 10/60; G16H 40/63; G16H 50/30

(57) ABSTRACT

A healthcare information management system for screening patients during medical consultations. Specifically, the system can be used in screening sites and comprises an integrated architecture for storing and receiving patient data. This architecture is integrated in a self-service station comprising peripheral devices for measuring vital signs such as arterial blood pressure, blood oxygen, body temperature, heart frequency and rhythm, weight, height and a spectrometer for quick pathology tests. The proposed solution enables patients to be classified (screened) automatically during preliminary healthcare in hospitals, clinics, doctor's offices or corporate outpatient clinics, by applying specific algorithms.

### FIELD OF THE INVENTION

The present invention relates to a healthcare information management system for screening patients before medical care. Specifically, the system can be used in the hospital area and comprises an integrated architecture for storing and receiving patient data. This is integrated with a service station comprising peripheral devices for measuring vital signs such as blood pressure, blood oxygenation and body temperature, heart rate, weight, height and glucose measurement.

The proposed solution allows the automatic classification of patients in pre-service (screening) in hospitals, health centers or corporate outpatient clinics through the application of specific algorithms.

### BACKGROUNDS OF THE INVENTION

One of the main challenges for the hospital field consists of organizing and screening the flow of patients based on their respective symptoms. Initially considered a typical emergency room phenomenon, it is now known that hospital overcrowding is a systemic problem.

In this scenario, the Risk Classification (CR) is a tool developed to optimize care in emergencies and identify patients who need priority in care and treatment, through a dynamic assessment process.

Among the most common tools is the Manchester Risk Classification System (MRCS) developed by healthcare professionals in the United Kingdom and consists of a guideline that establishes the priority of care in emergencies, prioritizing patients under greater risk clinical conditions. The methodology is based on the patient's main complaint, which leads to a flowchart of clinical conditions. Each flowchart contains discriminators that guide the investigation, generating a severity classification according to the provided answers.

This classification is described visually (colors) that indicate the maximum time interval for the first service; the red color denotes an emergency condition, for immediate service; the orange color distinguishes very urgent conditions, for service at intervals of less than 10 minutes; the yellow color suggests urgency (up to 60 minutes); those classified in green would be of little urgency and service could occur in up to 120 minutes; those colored blue, in turn, are considered non-urgent and their service is indicated to take between 120 and 240 minutes.

Although it is a crucial system for the organization of the emergency services, there are extremely limiting factors related to the waiting time between opening the service form and classification, which inevitably generates queues and worsens the patient's condition as a result of the delay in starting treatment.

In this sense, there are proposals including the use of kiosks to measure signs with specific peripherals to obtain vital signs such as blood pressure, heart rate, temperature, oxygen saturation. Optional elements include electrocardiogram, body weight and height.

Although relatively effective, this information, as well as clinical symptoms, is manually entered into systems or medical records by healthcare professionals.

Currently, the procedure consists of obtaining the patient's vital signs, questioning he/she about clinical symptoms and manually entering the information into the system to then carry out the classification according to the parameters of the chosen protocol. It should be noted that the execution of all the mentioned steps depends on a healthcare professional.

Additionally, it is noted that information related to vital signs is communicated to the patient and stored, without being used for screening systems or even for remote consultation by the doctor or healthcare professional. This fact creates serious inconveniences for the execution of the appropriate clinical protocol, as will be noted below.

Patent ES2575712, for example, describes a unit for capturing, storing and processing biomedical parameters comprising a main structure for access by the patient. Its internal area features a control panel with a screen and monitor configured for different measurement components, such as temperature and glucose. This system further requires a processor and a printer with a magnetic/RFID card reader and is restricted to a single communication with a central server. The absence of a specific architecture for integrating this data as well as the physical dimensions of the unit substantially limits its implementation in emergency systems.

Patent CA2751173 presents similar limitations. This document discloses a kiosk incorporating blood pressure measuring devices, pulse oximeters, electrocardiograms (EKGs and ECGs), glucose meters, and thermometers. In this case, the processor is coupled to the measurement devices and integrated with a database server configured for data communication using an HTTP protocol. The main disadvantage of this proposal is the lack of integration with medical records systems, information security (password management) or even with mobile applications for healthcare professionals and patients. As will be noted below, the present invention overcomes such disadvantages by means of specific data tokenization solutions.

A proposal that attempts to overcome the issue of physical space of the devices described above is disclosed in WO2019241867. This publication describes equipment for patient screening involving a cabinet comprising several vital signs measuring devices integrated with embedded software to execute the Manchester protocol. In this case, the information is merely printed, without any type of integration with other elements such as mobile platforms or remote servers. Such a fact makes this system extremely limited in outpatient emergency situations, which require precision in communicating information between healthcare professionals.

Publication WO2016151364 describes a patient care system comprising a device for administering medical treatment to a patient and a server configured to receive and transmit data from users (patients and healthcare professionals).

In this regard, the server system of WO2016151364 comprises a database configured to encrypt and store data related to patient care, an application server including patient care software components for disease management and patient information, and a communication server including a web application for transferring data over the internet. The components of the patient care architecture are configured to receive data from the medical device through the communications network, and process it together with patient data to generate a report for patient treatment. An expansion of this project appears to be described in WO2019073081, in which a system of care for a patient with chronic diseases is disclosed. This comprises a server system configured to receive and transmit data between patients and healthcare professionals, associated with a patient-related database and a database configured to store data related to predefined therapeutic interventions.

Both references WO2016151364 and WO2019073081 present architecture fundamentally configured to monitor patient adherence to the treatment regimen. No forms of integration with equipment or devices for measuring vital signs are described or suggested, much less proposals for integrating this information with the aim of automatically classifying patients in pre-service (screening).

Another approach is reported in document US2020211709 and consists of a care system generated from the patient's profile and history. It is a token-based system. User's questions are converted into words and the most relevant answers are mapped and selected from a corpus of data. The singular focus of this document is on medical consultations. US2020211709 does not contemplate or benefit from the inclusion of additional elements such as oximeters, temperature sensors or ECG equipment nor does it address the problem of organizing and screening patients.

An alternative solution is discussed in WO2016130532, in which there is described a communication system configured to receive electronic and oral communications from a patient, scan data to determine the patient's medical needs, and display information to medical staff. This will be able to advise the patient on the most appropriate form of medical assistance related to the identified symptoms.

The main limitation of WO2016130532 is precisely the lack of integration of the information obtained as well as the need to manually enter clinical conditions and/or vital signs into the user's device. The concept proposed in this reference does not provide an adequate solution for patients with more serious conditions in the emergency service, being merely a palliative to the problem of overcrowding.

KR20160131453 in turn describes a health management system for monitoring vital signs for diagnosis and remote medical control. In this project, a kiosk unit is connected to a server through a network. The unit is in communication with medical examination equipment, such as a body weight scale, a heart rate meter, a blood pressure meter, a glucose meter and the like. In this case, the unit device measures and compares health examination data with normal ranges, i.e. defined ranges, and determines the health status of a user.

The main disadvantage of KR20160131453 is the lack of constructive flexibility; this does not include additional devices such as thermometers or oximeters, much less features patient identification modules. The absence of patient identification modules such as QR code or magnetic card makes this project unsuitable for patient care and screening. This limitation is aggravated by the lack of settings for printing service passwords as well as the lack of customization of the clinical questionnaire by the patient (a limitation of its architecture-aimed specifically at tests).

Another limiting aspect is the patient's measurement history; KR20160131453 does not offer any integration with online platforms, with the result displayed by reading a QR code, susceptible to damage and failures in the integrity of the stored data. Such features practically make it impossible to apply this project in hospital institutions due to the lack of more flexible means for processing and storing data and screening patients.

### Objectives of the Invention

Given the number of variables involved, the system described by the present invention overcomes the disadvantages of the State of the Art, providing greater integration of data obtained by the self-service device, related to the flow of information and patient screening, in a more compact unit and integrating equipment for measuring vital signs such as blood pressure, blood oxygenation and body temperature through wireless communication. Some specific embodiments include communication via Bluetooth Low Energy (BLE), Universal Serial Bus (USB) and/or Wireless Fidelity (Wi-Fi).

Specifically, the present system overcomes the limitations present in the State of the Art through a system containing a unit comprising devices and meters of clinical parameters including thermometer, oximeter, pressure, weight, height, heart rate/rhythm and glucose meters in association with care management and patient screening modules.

More specifically, such modules comprise means for patient identification (including QR Code or magnetic card with bar codes) and means for printing care screening passwords and measurements.

The modularized architecture of the unit allows the implementation of online check-in and obtaining patient's measurement results via mobile applications, integration with the medical records of the hospital institution or corporate outpatient clinic and customization of the clinical questionnaire by the user.

Additionally, there is the tokenization of data preserving its integrity, inclusion of data in a dashboard in the backend system to visualize the day's service screening and all statistical/historical data. As will be noted, the system configuration of the invention allows for reliable and accurate integration of patient information for screening.

The self-service station features modular units such as blood pressure meters, oximeter, body thermometer, thermal printer, QR Code reader, magnetic reader, camera, touchscreen monitor and an application that helps guide the patient through self-service for screening and measurement of the vital signs, as well as generation of service password and appointment location. The architecture is based on a web system with a patient care classification panel based on the criticality of the measured signs and the order of arrival of patients, and also for configuring the customer service and management station.

This system in turn integrates with a mobile application that allows viewing of the care classification panel, the measurements of vital signs and patient data as well as a mobile application for the patient to view his/her data and the entire history of the measurements of his/her vital signs, as well as allowing online check-in at the establishment, describing his/her symptoms and speeding up his/her service. Finally, there is the interface for integrating patient data with the establishment's medical records, which allows the storage and processing of patient data for references and standardization of clinical protocols.

In a second embodiment of the invention, the present invention describes a screening station for medical care comprising said system.

### BRIEF DESCRIPTION OF THE FIGURES

FIG. 1 presents the flowchart of the method implemented by the outpatient screening system.

FIGS. 2 and 3 illustrate an embodiment of the station comprising a cabinet, preferably made of carbon steel. The cabinet accommodates peripheral devices such as blood pressure meter (103), cuff (104), oximeter (113), thermometer (109), thermal printer (112), QR Code reader (110), magnetic reader (111), camera (106), display device (108).

FIG. 4 is a front view of the Station, showing the height meter (106) and the scale (105).

FIG. 5 illustrates the architecture of the medical information management system for outpatient screening.

### DETAILED DESCRIPTION OF THE INVENTION

The proposed invention will be described below based on the presented Figures. It should be noted that the expression “an embodiment” means that a certain element, structure or feature is included in at least one embodiment of the present invention. Likewise, the expression “in a preferred embodiment” present in the specification does not necessarily contemplate embodiments or alternatives that are mutually exclusive to others.

The term “user” refers to any healthcare professional responsible for assisting the patient in interacting with the station and the described management system. The term “patient” in turn refers to any person who requires medical guidance or assessment.

The service station (100) will be discussed below, based on the presented figures.

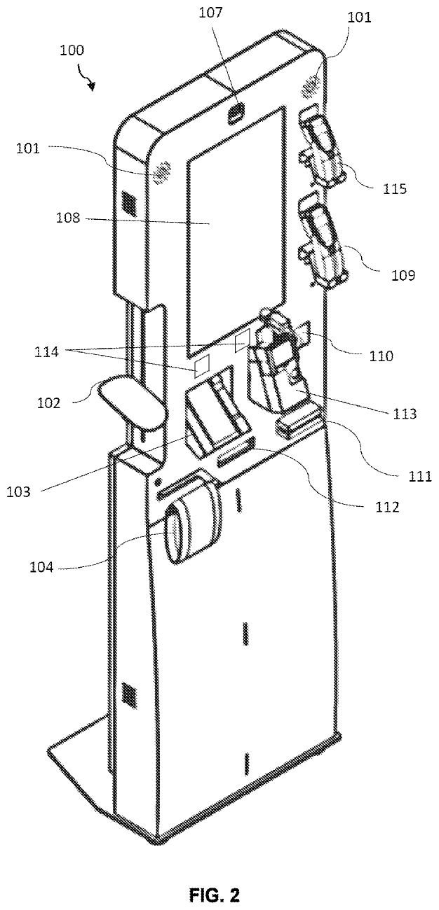

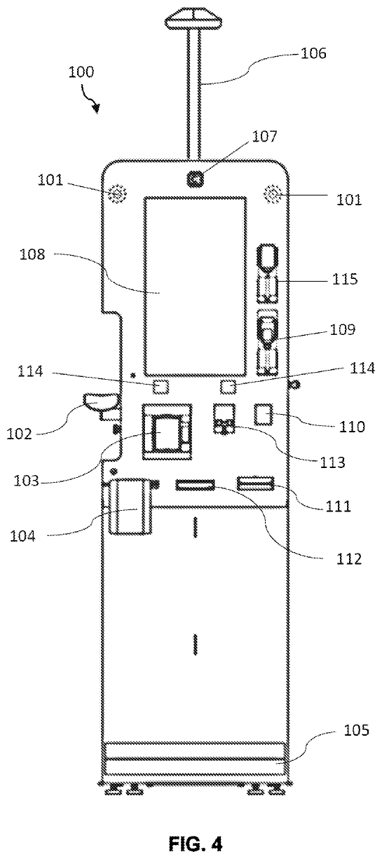

FIGS. 2 to 4 show the physical structure of the cabinet of the service station (100). For reference purposes, said cabinet will be described segmented into upper (A) and lower (B) regions or planes.

In one embodiment, the front surface of region (A) of the cabinet comprises: at least one pair of audio outputs (101) located in the front region that are lateral and opposite each other; a camera (107) disposed centrally between the pair of audio outputs (101); at least one ultrasonic height meter (106) located at the top of region (A); at least one armrest support (102); at least one blood pressure meter (103); at least one integrated display device (108); at least one thermometer (109); at least one oximeter (113); at least one QR code reader (100), a magnetic card reader (111) and a printer (112).

Region (B) of the cabinet in turn comprises a weight scale (105).

According to FIG. 2, the cabinet has a cut-out side profile, accommodating an armrest support (102). In one embodiment, said support (102) is located adjacent to the arterial pressure meter (103) while it is accommodated in a cavity on the front surface of region (A) in the cabinet.

At the top of region (A) there is an ultrasonic height meter (106), with volume and height indicators.

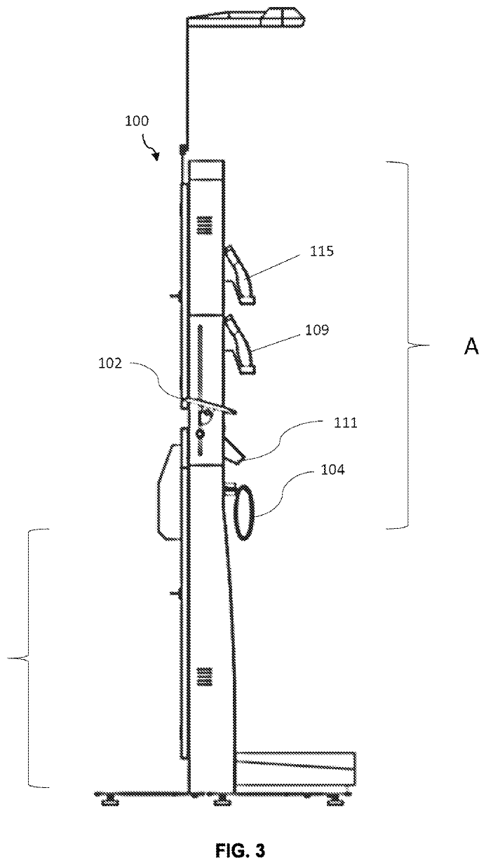

Considering FIG. 3, the rest support (102) comprises a platform for resting the arm, integrated with a rail; this allows to adjust the height and adapt the patient's positioning.

Considering the front region of the cabinet, there is a thermometer (109), as well as an output for audio connection. The oximeter (113), the magnetic card reader (111) and a printer (112) are located in the lower portion of the cabinet. In the lower region, there is a scale (105) attached to measure the patient's weight. The station (100) further includes an electrocardiogram device (114) and an infrared spectrometer (115) located in the cabinet.

The station also includes a data processor. The expression “data processor” encompasses all types of apparatus, devices, and machines for processing data, which includes by way of example a programmable processor, a computer, or multiple processors or computers. Preferably, the processor has a motherboard with an integrated Bluetooth Low Energy device. The apparatus may include a set of special-purpose logic circuits, for example, an FPGA (field programmable gate array) or an ASIC (application-specific integrated circuit). In an alternative embodiment, the processors may additionally include hardware, code that creates an execution environment for the computer program in question, e.g., code that constitutes processor firmware, a protocol stack, a database management system, an operating system, or a combination of one or more thereof. Non-exhaustive examples of operating systems include Windows system from Microsoft, Android from Google, iOS from Apple, Symbian from Nokia, Bada from Samsung Electronics, BlackBerry from BlackBerry, and Linux (Ubuntu). Preferably, the Linux operating system (Ubuntu) is used.

According to one embodiment of the invention, the processor is responsible for executing one or more instructions through the patient's interaction in the interactive environment.

The interactive environment consists of a user interaction interface, managed by an application to display and visualize the consultation content on the station or on mobile devices. The system further allows optimizing patient screening, as well as storing and sending test results to external servers in the cloud.

The application corresponds to a software code programmed to interact with hardware elements to carry out one or more measurement steps via communication protocol (preferably Bluetooth LOW Energy) and will be discussed later. Other functionalities include accessing, reading, updating and modifying data stored on the station or on external servers. Furthermore, the application provides a user interface for user interaction with the aforementioned hardware elements (peripheral devices). The user interface may include a Graphical User Interface (GUI), Application Programming Interface (API), and the like.

It should be noted that the application may be supported on any computer-readable media suitable for storing computer program instructions and data. These include all forms of memory, media, and non-volatile memory devices, such as, for example, semiconductor memory devices, e.g., EPROM, EEPROM, and flash memory devices; magnetic disks, e.g., internal hard disks or removable disks; optical-magnetic disks; and CD-ROM and DVD-ROM disks. Preferably, the application is stored on computers with a Linux operating system (Ubuntu).

The communication network in turn will be responsible for sending and receiving user commands and data. This allows the screening system to transfer information to the cloud, backend servers (200) and mobile devices (202). Communication network embodiments include 2G, 3G, 4G, 5G, Wi-Fi and similar technologies.

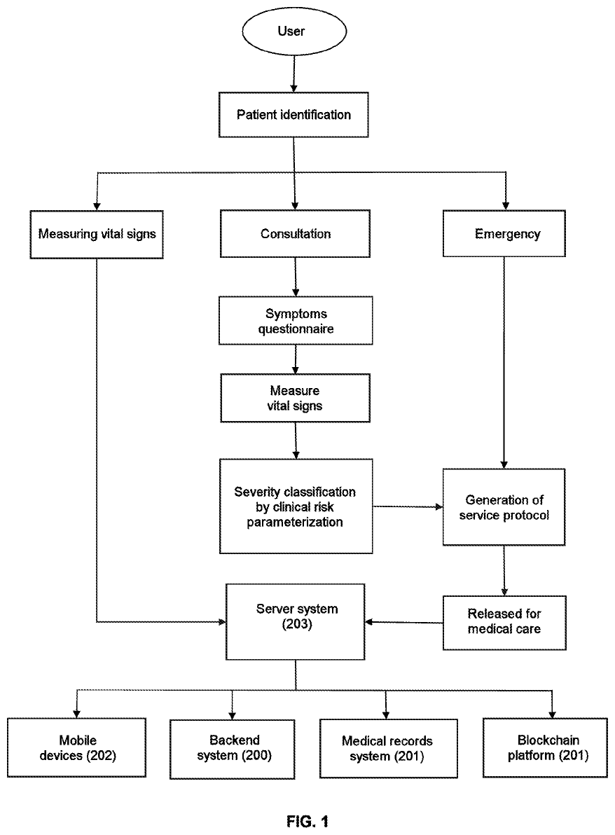

Specific embodiments of the medical information management system will be presented below (FIG. 1). The system elements are specifically configured to capture test results generated by the peripheral devices of the station (100).

To initialize the system, the user or patient enters his/her identification by reading an identity card on the QR Code reader (110) or data reader (111) configured to read the patient's identification data. In one embodiment, the element (111) is preferably adapted for reading the patient's medical insurance card. After identification, the user or patient inputs personal information for service in the station's user interaction interface or remotely, by using mobile devices (202).

The user interaction interface includes audio outputs (101), camera (107) and any display devices (108), preferably an LCD (liquid crystal display) monitor with capacitive or resistive touchscreen technology. In a preferred embodiment, the interface presents options for language selection by the user or patient.

Alternative embodiments of (108) comprise IPS-LCD, TFT-LCD, CRT (cathode ray tube) monitor for displaying information, keyboard and/or a pointing device, for example, a mouse or trackball, through which the user can provide inputs on the interface.

The mobile device (202) in turn relates to any communication device such as laptop, cell phones or tablets. In a preferred embodiment, the mobile devices are cell phones and tablets. The devices (102) may present an operating system such as Windows from Microsoft, Android from Google, iOS from Apple, Symbian from Nokia, Bada from Samsung Electronics and BlackBerry from BlackBerry. It should be noted that these are not limiting embodiments but rather illustrative. In embodiments of the present invention, the application operates on any mobile device with any version of the aforementioned operating systems. Preferably, Android 9 and iOS 10 (minimum versions) are used.

In a preferred embodiment, data will be sent to at least two mobile devices (202)—that of the patient and that of the healthcare professional. Both will be able to view the history of the measurements of the vital signs, in addition to allowing online at the establishment, describing symptoms and optimizing the service.

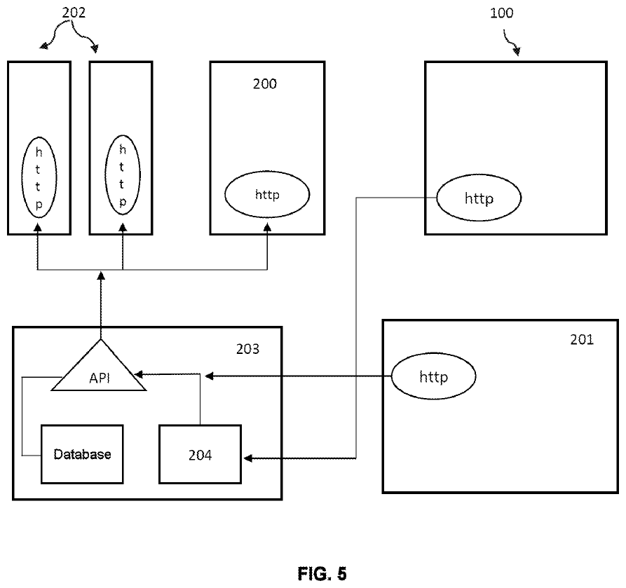

Considering FIG. 5, after the identification step, feedback information is displayed in which the user must confirm his/her personal data. It should be noted that the feedback information provided to the user can be any form of sensory feedback information, for example, visual feedback information, auditory feedback information, or even tactile feedback information.

After confirming the patient's data, the display device (108) will request a consultation from the user comprising the station's operating mode options:
i) Consultation: the user or patient must report his/her symptoms and measure his/her vital signs to classify the service;
ii) Emergency: sending instructions to take the patient directly to the doctor's office;
iii) Measuring Vital Signs: in this embodiment, the patient will measure vital signs by using peripheral devices, whose data will be stored and sent to the patient's application.

The user selects the desired mode of operation, which in turn will generate an instruction in response to the consultation. The instruction will display a specific and detailed user interface later.

### Consultation Mode

When selecting the “Consultation” operating mode, the interface generates a questionnaire replicating a simplified anamnesis. Questions and answer types can be configured and customized by the System Administrator.

In the preferred embodiment, the interface generates a question regarding the symptom, the duration of the symptom and drug allergy, in which the user must provide instructions by means of the device (108). Instructions may be provided sequentially or in a single step. In a preferred embodiment, instructions are provided in sequential interfaces (description of symptoms, followed by the duration thereof).

More preferably, the duration of the symptom is informed using the options: “Less than 1 day”; “1 to 3 days” or “More than 3 days”.

More preferably, drug allergy can be reported using the options “Acetylsalicylic acid (AAS)”; “Dipyrone”; “Others”. The “Others” option will provide a text field for the user or patient to enter the name of the drug. It should be noted that these fields are fully configurable and can be adapted according to the establishment's needs.

Questions can be configured from the station management web module. The “consultation” mode can be ended by the patient or user. In this case, a service protocol will be generated (password and office). The user will proceed to the “measuring vital signs” mode, discussed below.

### Emergency Mode

In this embodiment, the user, upon verifying the criticality of the patient's clinical condition, sends an instruction to the Station's processor. This in turn will generate a service protocol to take the patient directly to the doctor's office and, by using the printer (112), results can be physically printed, if necessary. The Station includes an Application Programming Interface (API) for integration with the establishment's password management and service location system.

### Measuring Vital Signs Mode

After answering the questionnaire in the “Consultation” operating mode, continue with the “Measuring Vital Signs” operating mode. In this event, the user interaction interface is configured for the use of the peripheral devices. In a preferred embodiment, the interface provides audiovisual resources to instruct the user how to use each of the peripheral devices of the Station.

The first measurement is blood pressure. After instruction from the patient, the user interface activates the blood pressure meter (103).

The second measurement is body temperature, carried out by the thermometer (109).

The third measurement is blood oxygenation carried out by the Oximeter (113).

All results will be stored in the server database and sent to mobile devices (202) and web systems via http protocols.

The measurements include electrocardiogram examination by using the electrocardiogram (ECG) device (114), height meter (106), scale (105) to measure the patient's weight and infrared spectrometer (115) for molecular assessment of quick-result glycemic index. Infrared radiation interacts with the sample, generating spectra according to certain chemical groups (“fingerprint”) and enables the molecular identification of pathologies and systemic conditions (such as glycemia, for example).

Based on the results provided by peripheral devices, the application assesses the patient's criticality condition by using a clinical risk parameterization protocol. It should be noted that there can be used any types of severity classification protocols by risk parameterization.

By using the adopted severity classification protocol algorithm by risk parameterization, the application generates a classification of the patient's care priority by colors, assigning them according to the care priority. An example of classification can be assigning the “priority 1—Emergency” scale (immediate service); priority 2—Very Urgent (service within 10 minutes); priority 3—Urgent (service within 1 hour); priority 4—Little Urgent (service within 2 hours); and priority 5—Non-Urgent (service within 4 hours).

The station (100) will transmit test information and patient data to a server system (203), configured to receive and transmit data over a communication network including patients and healthcare professionals. Preferably, the server (203) is in the cloud.

The server system (203) comprises at least one messaging server (204), a database and an application programming interface (API).

The data may be stored in the server database (203). Non-exhaustive embodiments include multi-platform software such as SQL Server 2019™ and MongoDB™. Preferably, MongoDB™ is used.

The messaging server (204) is responsible for handling message traffic. Preferably, the messaging system is implemented to support Advanced Message Queuing Protocol (AMQP) messaging protocol such as RabbitMQ™.

Next, the data is tokenized and sent to a cloud data tokenization platform and to integrate patient data with the establishment's medical records system (201). The system (201) automatically makes the information available to any medical records management system.

In parallel, the API integrates data with backend systems (200) and mobile devices (202), generating a care classification panel based on the criticality of the measured signs and the order of arrival of patients. The data is made available to the outpatient team by the healthcare professional and the patient himself/herself.

The backend system (200) also includes tools for configuring the customers service and managing station, users, access profiles, patients, questionnaires and screening station units.

In an optional embodiment, the application can access one or more databases. These can be from medical institutions, hospitals and the like with the purpose of collecting data related to the patient's health.

Tokenization comprises the conversion of the sequence of results from the user's or patient's consultation into tokens, in real time. This functionality guarantees the integrity of information in the system of the present invention. In a preferred embodiment, the method employs blockchain tokenization.

A blockchain is a data structure that can be used to implement tamper-resistant records. Multiple nodes follow a common protocol in which the customer transactions are packaged into blocks and the nodes use a consensus protocol to agree on the next block. The blocks carry cumulative cryptographic hashes, making it difficult to tamper the ledger.

In a preferred embodiment, the Hyperledger Fabric Blockchain platform is used.

It should further be noted that the system offers alternative embodiments of use. Considering the autonomous nature of the system, it is possible to adapt structures (107) and (108) to screening patients for in-person and virtual consultations as well as virtual care. Thus, other possible embodiments of use of the invention are patient care through teleconsultation, interpretation of medical exams buy means of telediagnosis, telemonitoring, among other activities that can be carried out remotely. At the end of the assessment of the vital signs, patient care can be directed to a telemedicine platform.

It should be noted that the embodiments for using the system described above are not exhaustive-other possibilities can also be found, without departing from the scope of the present invention.

1. A patient self-service station for hospital and outpatient screening comprising a cabinet comprising an upper (A) and a lower (B) region;
wherein the lower region (B) of the cabinet comprises a weight scale (105);
wherein a front surface of the upper region (A) of the cabinet comprises:
at least one pair of audio outputs (101) located in the front region that are lateral and opposite each other;
a camera (107) disposed centrally between the pair of audio outputs (101);
at least one ultrasonic height meter (106) located at the top of the upper region (A);
at least one armrest support (102);
at least one blood pressure meter (103);
an integrated display device (108);
at least one thermometer (109);
at least one oximeter (113);
at least one data reader (110; 111);
a printer (112);
an electrocardiogram (ECG) device (114);
and infrared spectrometer (115);
said cabinet being provided with a cut-out external side profile, accommodating an armrest support (102), said support (102) located adjacent to the blood pressure meter (103), said meter (103) accommodated in a cavity on the front surface of the upper region (A); and a data processor located internally to the cabinet.

2. The station according to claim 1, wherein the integrated display device (108) is selected from the group consisting of an LCD monitor with capacitive or resistive touchscreen technology, IPS-LCD, TFT-LCD and cathode-ray tube monitor.

3. The station according to claim 1, wherein the data processor includes a motherboard that contains a Bluetooth Low Energy, Universal Serial Bus and Wireless Fidelity device.

4. The station according to claim 1, wherein the data processor is selected from a programmable processor, a computer, or multiple processors or multiple computers.

5. The station according to claim 1, wherein the armrest support (102) is adjustable in relation to height.

6. A medical information management system comprising a self-service station, as defined in claim 1 comprising:
a server system (203) configured to receive and transmit data over a communication network;
said server system (203) comprising at least a database, an application programming interface and a messaging server (204);
said server system (203) in communication with at least one backend system (200);
said server system (203) in communication with at least one mobile device (202);
a medical information management application configured to provide the user with options to:
i) report symptoms and measure the patient's vital signs;
ii) send instructions to take the patient directly to the doctor's office; or
iii) measure vital signs using the peripheral devices of the station (100);
wherein patient data captured from the peripheral devices of the station (100) is tokenized and sent to an application programming interface on the server (203); and
integrating with the establishment's medical records system (201).

7. The system according to claim 6, wherein the messaging system (204) is implemented to support the Advanced Message Queuing Protocol (AMQP) messaging protocol.

8. The system according to claim 6, wherein the application programming interface generates a service classification based on the criticality of the measured signs and the order of arrival of patients.

9. The system according to claim 6, wherein the application accesses one or more databases.

10. The system according to claim 6, wherein the tokenization step employs blockchain tokenization.

11. The system according to claim 6, wherein the data captured by the peripheral devices of the station (100) relates to measurements of height, weight, body temperature, blood pressure, and pulse rate.

12. The system according to claim 8, wherein the service classification uses a severity classification by risk parameterization.

13. The system according to claim 6, wherein the mobile device (102) is a laptop, cell phone or tablet.

14. The system according to claim 13, wherein the station (100) integrates with the establishment's medical and the records system (201) establishment's password and service location management system.

15. The system according to claim 6, wherein said system automates screening for medical appointments, monitors vital signs and is applied in telemedicine.

16. A computer-readable means comprising a set of instructions that, when executed, carry out the method with the steps of:
a) measuring the patient's vital signs using the peripheral devices of the station (100);
b) tokenizing patient data captured from the peripheral devices of the station (100), via the blockchain platform;
c) sending to an application programming interface on the server (203); and
d) integrating with the establishment's medical records system (201) and the establishment's password and service location management system.
